(19) United States
(12) Patent Application Publication
(10) Pub. No.: US 2009/0305336 A1
(43) Pub. Date: Dec. 10, 2009

(54) DEVICES AND METHODS FOR SAMPLING MICROBIAL FLORA

(75) Inventor: Gary Swanson, Elkhorn, WI (US)

Correspondence Address: WHYTE HIRSCHBOECK DUDEK S.C.;INTELLECTUAL PROPERTY DEPARTMENT, 33 East Main Street, Suite 300, Madison, WI, 53703-4655, US

(73) Assignee: GENEVA LABORATORIES, INC., Elkhorn, WI (US)

(21) Appl. No.: 12/479,287

(22) Filed: Jun. 5, 2009

Related U.S. Application Data
(60) Provisional application No. 61/060,338, filed on Jun. 10, 2008.

(51) Int. Cl. C12Q 1/04; C12M 3/00

(52) U.S. Cl. 435/34; 435/289.1; 435/305.2

(57) ABSTRACT

Methods, devices and kits for sampling for a microorganism or microbial population on the digits of the extremities of an individual are described. In embodiments of the invention, the device comprises a container, such as a cell culture plate, with a growth medium that is delineated into multiple quadrants, which can be visualized through the growth medium, to aid in the placement of a digit within a designated location on the growth medium.

### CROSS REFERENCE TO RELATED APPLICATIONS

This application claims benefit under 35 U.S.C. § 119(e) of U.S. Provisional Application No. 61/060,338 filed Jun. 10, 2008, which is incorporated herein by reference as if set forth in its entirety.

### FIELD OF THE INVENTION

Embodiments of the invention relate to devices, methods and kits for sampling for a microorganism or microbial population on an extremity of an individual. The methods, devices and kits of the present invention provide a rapid and efficient manner to monitor the extremities of individuals working in restaurants, hospitals and in other such environments where workers are exposed to microorganisms.

### BACKGROUND OF THE INVENTION

A principle of microbiology is that pathogens on the hands, which can be transferred to other body parts such as the mouth, nose and eyes, are a primary cause of infectious disease in humans. The actual scope of the damage caused by infections resulting from hand-borne pathogens is generally less known. Up to eighty percent of all infections ranging from benign colds and more debilitating flu to the deadly Ebola virus infection, can be transmitted by touch. The average American will contract two to four cold or flu infections in a typical year, and experience up to four to six significant gastrointestinal disruptions during the same period. Colds alone account for an annual loss of 300 million person days of work and school annually. Illnesses associated with the flu virus cost Americans $10 billion a year in lost wages and medical expenditures in addition to the 20,000 to 50,000 deaths each year from complications of influenza infections. For years the famed Mayo Clinic has used the following slogan in an attempt to drive home a point about the need for effective hand hygiene to combat serious diseases: “The ten worst sources of contagion are our fingers.”

Each year more than 2 million hospital-acquired or nosocomial infections occur in the United States, costing some $4.5 billion in additional charges. The Centers for Disease Control estimates more than one-third of healthcare associated infections can be prevented through better infection control programs of which hand cleaning and efficient methods for testing hands for contamination are the centerpiece for reducing the spread of infection. Hospitals are only one of many organizations burdened with hand-borne disease costs. Other organizations and occupations with similar issues include restaurants, food handlers, and others working in a clean or sanitary environment that are in contact with food or other sensitive products.

Epidemiologists commonly search for microbial contamination in the environment as well as in food products to find common causes of outbreaks. Disease promulgated through food-borne pathogens can be particularly devastating as the disease may be spread quickly and infiltrate many different geographic regions. If a food worker handles contaminated items, and subsequently handles additional items or products, the contamination can spread quickly to other items and products. One example is the outbreak in 1992 of enterohemorrhagic E. coli (EHEC) in the Pacific Northwest of the United States due to contaminated ground beef. EHEC is a relatively “newly discovered” pathogen. EHEC was first isolated in 1975, and it was not until 1982 that E. coli 0157:H7 was associated with two food-related outbreaks of hemorrhagic colitis in the United States. EHEC is particularly virulent and can trigger deadly complications, including severe abdominal cramps and acute renal failure in children as well as cardiovascular and central nervous system problems.

As another example, Salmonella, a Gram-negative bacterium, is a leading cause (more than 50%) of total bacterial food borne disease outbreaks, according to the United States Centers for Disease Control (CDC) surveillance of food-borne diseases. Salmonella can infect a broad variety of warm- and cold-blooded animals, and can survive for long periods of time outside a host.

A third example is Listeria, a genus of Gram-positive bacteria, which is widely distributed in nature, having been isolated from soil, water, vegetation and many animal species. The genus Listeria contains seven identified species: L. monocytogenes, L. innocua, L. ivanovii, L. seeligeri, L. welshimeri, L. grayi, and L. murrayi. Virulence of these species is quite diverse and L. monocytogenes is considered to be the only pathogenic species for humans. Serious outbreaks of human listeriosis have not been frequent, but have been identified with increasing incidence. For specific outbreaks of listeriosis, estimates place mortality at 30% to 40% of affected patients, however, little is known of the minimum infective dose. One particularly troublesome aspect of Listeria control in foods is that Listeria can grow at temperatures as low as −0.4° C. and as high as 44° C. These factors all contribute to the increasing significance of Listeria as a food pathogen.

It would be useful to provide devices, methods and kits for monitoring for the presence of microbial contamination that overcame these problems.

### SUMMARY OF THE INVENTION

The invention relates to devices, methods and kits for assaying or testing for contamination, including microbial contamination on the extremities of an individual. Embodiments of the invention comprise a device for sampling for the presence of a microorganism or microbial population on an extremity of an individual composed of a growth medium within a container, wherein the growth medium is delineated into quadrants that can be observed through the growth medium. The quadrants can be defined, for example, by markings on a sheet of paper or label attached to the bottom surface of the container of the device. For example, markings on the sheet can delineate the medium into ten quadrants, with depictions of a right hand defining five quadrants and depictions of a left hand defining another five quadrants, which provides a systematic layout for testing each digit of each hand of an individual. The device can be used to test bare hands, or hands covered with an article including but not limited to gloves.

The present invention also relates to methods for testing for a microorganism or microbial population on an extremity of a subject individual. An embodiment of a method for testing for a microorganism or microbial population on an extremity, for example a hand, comprises placing one digit of a hand onto the surface of a growth medium contained within a device, with the digit placed within a marked or otherwise delineated quadrant of the growth medium; rolling the digit (e.g., the fingertip) on the surface of the growth medium, e.g., in a direction from left to right; rotating the device so that the remaining regions are oriented along an edge of the table toward the individual; performing a “finger print roll” on the surface of the growth medium with each remaining digit of the hand within a single defined quadrant of the medium; returning the device to the original position (orientation) along the table; sampling a digit of the second hand; rotating the device so that the remaining regions are orientated along the edge of the table toward the individual; performing a “finger print roll” with each remaining digit of the second hand in contact with the growth medium within a separate quadrant; and sealing the device and labeling with the appropriate information.

The invention further relates to a method for preparing a device that can be used for testing for a microorganism or microbial population on an extremity, including but not limited to the digits of the hand. An embodiment of a method for preparing a device for testing for a microorganism or microbial population of an extremity, for example digits of a hand, comprises pouring plates with an appropriate type of growth medium such that digit placement quadrants or cells can be seen through the media, and labeling and packaging the plates.

The invention also relates to a kit for testing for microbial contamination. An embodiment of a test kit according to the invention is composed of a sealed container comprising growth medium delineated into multiple quadrants, and a schematic that can be observed through the growth medium, and illustrates where each article for testing (including but not limited to a digit of a hand) should be placed, and instructions for use of the kit, which can also include instructions for shipping and handling of the kit.

As evidenced by the continued outbreak of microbial diseases, a need exists for a mechanism for monitoring for microbial contamination, and in particular, a mechanism for monitoring the extremities of workers who are often in contact with the source of contamination. A key feature to prevent the promulgation of such diseases are methods devices or kits for monitoring the presence of such contamination on an extremity, particularly on the digits of the hand.

Embodiments of the invention provide devices, methods and kits that rapidly and efficiently assay for microorganisms, including bacteria, yeasts, molds, fungi, parasites and viruses, require no special or technical equipment, and can be performed in the field (on site). Embodiments of the present invention provide the ability to monitor for the presence of microbial contamination quickly and easily and can be used to reduce the risk of human infection and potential mortality.

Embodiments of the present invention comprise a device for sampling for the presence of a microorganism, which includes a growth medium, wherein the growth medium is delineated into quadrants and the quadrants can be observed through the growth medium. Embodiments of the present invention also comprise a method for sampling for the presence of a microorganism, comprising: placing a digit of a first hand from an individual on a surface of a growth medium in a quadrant of a container, wherein the bottom surface of the container comprises a sheet with a grid or layout that delineates the growth medium into said quadrants; and rolling the digit from left to right. Embodiments of the present invention also relate to a kit comprising a device comprising a container with growth medium, a sheet that can be coupled to the bottom of the container comprising a visible layout or grid that delineates the growth medium into quadrants, and instructions for use of the device.

### BRIEF DESCRIPTION OF THE DRAWINGS

Embodiments of the invention are described below with reference to the accompanying drawings, which are for illustrative purposes only. Throughout the following views, the reference numerals will be used in the drawings, and the same reference numerals will be used throughout the several views and in the description to indicate same or like parts.

FIG. 1 is a schematic perspective view of an embodiment of a device according to the invention.

FIG. 2 is an exploded view of the device of FIG. 1.

FIG. 3 is a top plan view of the device of FIG. 1, with an embodiment of a sheet delineating quadrants that are visible through a growth medium.

FIG. 4 is a plan view of the bottom surface of the device of FIG. 1 showing an embodiment of an instruction sheet attached to the device.

### DETAILED DESCRIPTION OF THE PREFERRED EMBODIMENT

The following description with reference to the drawings provides illustrative examples of devices and methods according to embodiments of the invention. Such description is for illustrative purposes only and not for purposes of limiting the same.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub-ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, the number of quadrants in each device, and the temperature of incubators.

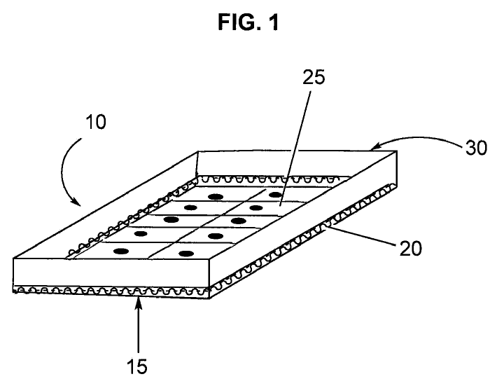

Embodiments of the invention comprise a device for sampling for a microorganism or microbial population on an extremity of an individual (e.g., finger, toe, hand, foot, etc.). An embodiment of a device (10) according to the invention is illustrated in FIGS. 1-4. The device (10) as depicted in FIG. 1 comprises a container (15), a layer of microbial growth medium (20), a sheet (25) that is visible through the growth medium (20), and a lid (30) to seal the container.

Any type of container that is suitable for a growth medium including but not limited to a plastic tray, a cell culture plate, and a bacterial culture plate may be used. The growth medium of the present invention can be any growth medium suitable for the growth of a microbial organism including but not limited to bacteria, viruses, fungi, protozoa, molds, yeasts, phage, archeabacteria and mycoplasm. The growth medium includes but is not limited to medium for bacteria, medium for fungi, nutrient medium, lysogeny medium, minimal medium, supplemented minimal medium, tryptic soy agar, eosin methylene blue, MacConkey agar, mannitol salt agar, X-gal agar, YM (yeast, mold) agar, blood agar, Hekotein enteric agar, xylose lysine desoxyschloate, and buffered charcoal yeast extract agar

In some embodiments of the invention, the growth medium comprises a selectable agent including a antibacterial(s), an antifungal(s), an antibiotic(s) and an agent(s) or compound(s) with an amino acid deficiency. An antibacterial includes but is not limited to penicillins, cephalosporins, erythromycin and erythromycin derivatives, sulfa drugs, trimethoprim, sulfamethoxazole, nitrofurantoin, aminoglycosides, polymyxin, quinolones, ciprofloxacin, and tetracyclines. In some embodiments, the growth medium comprises an antifungal agent including but not limited to nystatin and azoles. A plurality of devices can provide the growth medium as a serial dilution of a selected agent such that bacteria, fungi, etc. can be enumerated.

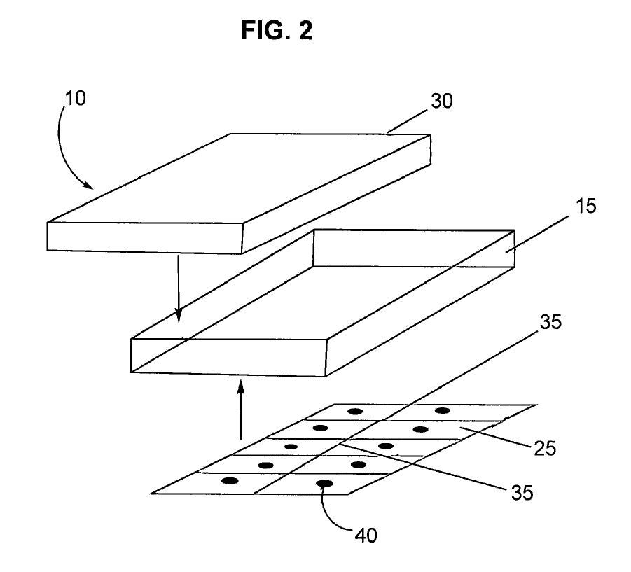

As shown in FIG. 2, in some embodiments, the sheet (25) can be affixed to the bottom surface of the container (15), wherein the sheet (25) comprises a grid or layout (35) to delineate the growth medium into quadrants, regions or cells. The sheet can be a label or a similar attachable medium that can be affixed to the bottom surface of the container (15) (e.g., cell culture plate, plastic tray, etc.) wherein the grid or layout is readable through the media.

In another embodiment, the sheet (25) can be placed on the bottom surface of the container (15), with the medium applied to (e.g., poured over the sheet). In this embodiment, the sheet (25) can be made of a material that can be sterilized, for example by heat or by ethylene oxide applications.

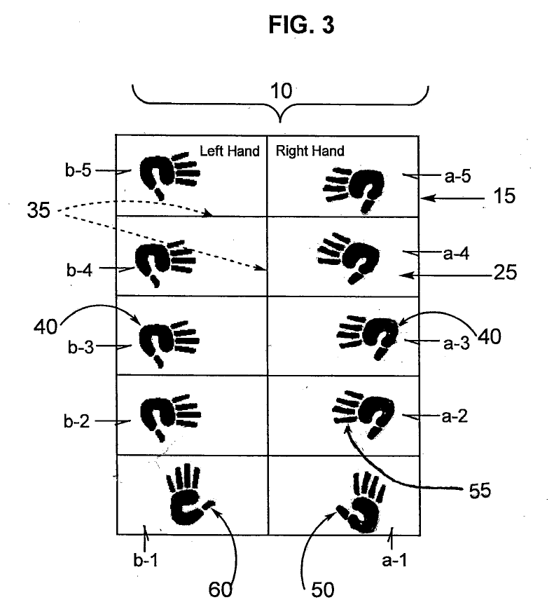

As shown in FIG. 3, the sheet (25) may comprise a grid or layout (35) to delineate the growth medium into ten quadrants, regions or cells, and can contain a symbol (40) to indicate the placement of each digit of each hand. As shown in FIG. 3, the symbol (40) can be a depiction of a hand. In other embodiments, the symbol can be a single digit of a hand, or a written description of a digit to be applied to the growth medium with the particular quadrant.

In other embodiments, the sheet (25) may comprise a grid or layout (35) to delineate the growth medium into two or more regions, quadrants, or cells (e.g., 2-10, 2-20, 2-50, 2-100, etc.). Each distinct region, quadrant or cell can contain the same medium or a different media. For instance, if the device comprises 20 regions, then there are numerous combinations for the type of medium within each region including but not limited to (a) ten regions containing one medium and the other ten containing a second medium; (b) all 20 regions containing different media; (c) five regions containing one medium, five regions containing a second medium, five regions containing a third medium, and five regions containing a fourth medium; and (d) fifteen regions containing one medium and five regions containing a second medium, etc. One skilled in the art will recognize that there are a plethora of growth mediums available, and that the distribution of each type of growth medium in each region can be determined based on the particular needs of the sampling protocol.

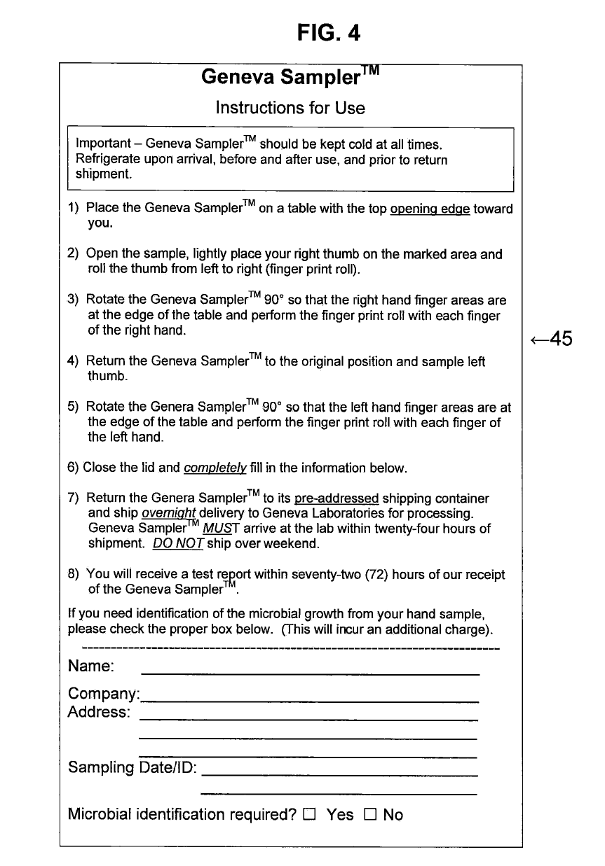

The sheet (25) can also comprise a set of instructions or directions (45) on the opposite side of the sheet (35) bearing the grid or layout. The sheet (45) can be adhered to the sheet (25) such that the layout or grid (35) for the placement of digits faces toward the growth medium (20) and can be seen through the growth medium (20) and the instructions on the sheet (45) face outward. As depicted in FIG. 4, the instruction sheet (45) can comprise instruction on how to use the device, and instruction on the appropriate procedures for handling and shipping the device.

The instruction sheet can comprise any words in any language to convey proper use of the device. One example of words that can be included on the instruction sheet are as follows: (1) Place the Geneva Sampler on a tale with the top opening edge toward you; (2) Open the sampler and place your right thumb on the marked area and roll the thumb from left to right (finger print roll); (3) Rotate the Geneva Sampler 90° so that the right hand finger areas are at the edge of the table; (4) Perform the finger print roll with each finger of the right hand; (5) Return the Geneva Sampler to the original position and sample left thumb; (6) Rotate the Geneva Sampler 90° so that the left hand finger areas are at the edge of the table; (7) Perform the finger print roll with each finger of the left hand; (8) Close the lid and fill in the information below; (9) Return the Geneva Sampler to its pre-addressed shipping container and return to Geneva Laboratories for processing; (10) You will receive a test report within seventy-two (72) hours of our receipt to the Geneva Sampler; (11) If you need identification of the microbial growth from your hand sample, please check the proper box below. (This will incur an additional charge.)

The instruction sheet may also include an area to provide the name of the individual to be tested; the name of the company; the address of the company; the sampling date and identification number; and an area to indicate the type of testing to be performed, including but not limited to bacterial, fungal or bacterial and fungal testing.

The device may also comprise indications for the use of the device in any words in any language. One such example, which should not be construed to limit the invention, is as follows: The Geneva Sampler is recommended for use in testing bare hands or gloved hands. The Geneva Sampler is an excellent method of documenting hand sanitation for hospital employees, food handlers, clean room employees or anyone working in a clean or sanitary environment in contact with food or other sensitive products. Hand sampling may be performed at the beginning or at the end of the work period, depending upon the purpose and how the information is to be presented. The Geneva Sampler is also an excellent problem solving tool. Hand sampling/documentation is recommended either weekly or monthly depending upon the sensitivity of the sanitary environment.

In embodiments of the invention, the device (10) can be structured and used for sampling, assaying or testing for the presence of any microbial organism including, but not limited to, bacteria, aerobic bacteria, anaerobic bacteria, microaerophilic bacteria, viruses, fungi, protozoa, molds, yeasts, phage, archeabacteria and mycoplasm.

In some embodiments, the device (10) can be used to sample or assay digits on the extremities of a single individual at a single time point or on the same individual at multiple time points including but not limited to sampling at the beginning of a work period and sampling at the end of a work period. In other embodiments, the device (10) can be used to sample or assay digits on the extremities on multiple individuals at a single time point or at multiple time points.

In another embodiment, the device (10) can be used for documenting hand sanitation for hospital employees, food handlers, fast food workers, restaurant employees, food processing employees, pharmaceutical plant employees, clean room employees, or other person(s) working in a clean or sanitary environment in contact with sensitive products.

Embodiments of the invention relate to a method utilizing a device (10) to determine the existence of a microbial organism on the extremities of an individual, including but not limited to, bare hands, covered hands, the right hand, the left hand, bare feet, digits of a bare or covered hand or foot or individual digits (e.g., index finger, middle finger, ring finger, little finger and thumb). In other embodiments, the invention relates to a method utilizing a device (10) to determine the genus and species of a microorganism or microbial population present on the extremities of an individual.

In other embodiments, the present invention provides a method for sampling a digit, including but not limited to the digits of a hand, for a microorganism or microbial population. The method can be used to sample a single digit or multiple digits. The method can be used to obtain samples from the right hand, the left hand, or both, and in any sequence. In some embodiments, methods of the invention can be used to enumerate the microbial flora present on a digit.

An embodiment of a method of obtaining a sample from a subject individual is now described with reference to FIG. 3, which should not be construed to limit the scope of the invention. A device (10) according to the invention can be placed on a surface, such as a table, with the top opening edge of the device (10) oriented toward the individual. The device can be opened and a digit, e.g., the right thumb (50) of the individual can be placed on the surface of the growth medium (20) within the marked area (a-1). In some embodiments, the digit can be rolled, for example, from left to right (finger print roll) over the surface of the growth medium (20). The device (10) can then be rotated about 90° (clockwise) so that the right hand finger quadrants (a-2 to a-5) of the device can be positioned along the edge of the table. Contact (e.g., a finger print roll) can then be made with the index finger (55) of the right hand on the growth medium (20). Each remaining digit of the right hand can then be sampled. The device can be returned to the original position and the left thumb (60) can be sampled. The device can then be rotated about 90° (counterclockwise) so that the left hand finger areas (b-2 to b-5) are along the edge of the table. Contact (e.g., a finger print roll) can then be made with each remaining digit of the left hand on the growth medium (20). The lid (30) of the device (10) can be affixed and individual-specific information can be placed on the appropriate label. Individual-specific information can include the name of the individual, the individual's birth date, the name of the company that the individual works for, the address of the company, the date the sample was taken, the location where the sample was taken, whether or not microbial identification is required, and any additional information that the user of the device may deem appropriate.

The device (10) can then be placed in a pre-addressed shipping container and returned to the appropriate laboratory for testing. Once received by the laboratory, the device (10) can be placed in an incubator of the appropriate temperature, typically about 25-50° C. In some embodiments, testing can involve solely observing for microbial growth. In other embodiments, testing can include determining the genus and species of the microbes(s).

The device (10) can be incubated on the premises where the sample was taken and, if growth is detected, the device can be sent off-site to a diagnostic lab for further analysis.

Embodiments of the invention also relate to a method for preparing a device (10) for sampling a microorganism(s) on extremities, including but not limited to the digits of the hand. In some embodiments, the method comprises sterilizing a container (15) of choice, for example, a plastic tray or a cell culture plate, among others, for containing the growth medium (20). The container (15) can be wrapped in a material that is permeable to a sterilizing gas or other medium. For example, the container can be wrapped in a double layer of central supply room (CSR) wrap, which is typically formed of a non-woven polypropylene of single or laminated layers, such as SMS (spunbond-meltblown-spunbond) and is permeable to sterilizing vapors yet impermeable to potentially contaminating microorganisms. CSR wrap is available from numerous suppliers, for example, Delasco (Council Bluffs, Iowa) and Viscot Medical, LLC (East Hanover, N.J.).

The wrapped container (e.g. trays) can be secured with ethylene oxide (EtO) indicator tape, and placed in a second container. The second container can then be sterilized, for example, by steam, ethylene oxide sterilization, gamma radiation, or other suitable methods known in the art.

The method further comprises pouring a growth medium into the sterilized container (15). The growth medium (20) can be prepared following the manufacturer's instructions for the specific media, e.g., Tryptic Soy Agar (TSA). The growth medium (20) can then be poured into the container (15) and cooled before placing the lid (30) on the container (15).

A sheet (25) or other suitable medium comprising a grid, for example, can be adhered to the bottom surface of the container (15). In the embodiment illustrated in FIG. 3, the sheet includes one side with a grid that divides the medium into ten quadrants, wherein five quadrants contain a depiction of the right hand and five quadrants contain a depiction of the left hand and an opposite side with instructions for use of the device (10). The sheet (25) can be adhered such that the grid faces toward the growth medium (20) and can be seen through the growth medium (20) and the instructions face outward. The sheet (25) can be secured to the container (15) with tape, adhesives or any other suitable material.

A label (not shown) can also be attached to the container (15) and or lid (30) to include a description, date, lot number, date of expiration, instructions for storage, etc.

The labeled device can be placed into an individual bag or other container. The packaged devices can be stored under refrigerated conditions until needed.

Embodiments of the invention relate to a kit for sampling or assaying for a microorganism or a microbial population on the digits of the extremity of an individual. In some embodiments, the components for performing the methods of the present invention can be packaged as a kit. For instance, an embodiment of a kit can include a device (10) comprising a lidded container (15) with growth medium (20), a sheet (25) coupled to the bottom surface of the container (15) comprising a visible layout or grid that delineates the growth medium into quadrants, regions or cells, and instructions (45) for use of the device (10) coupled to the sheet (25) and/or the bottom surface of the container. The kit can further include a pre-addressed envelope and/or label for shipment of the device (10) to a testing facility. In another embodiment, the device (10) and the sheet (25) comprising the grid or layout can be separately packaged. The kit can include multiple sheets (25) with several different grids, thereby allowing the end-user to choose the sheet that best suits the needs of the testing occasion. Another embodiment of a kit can include appropriate buffers and solutions to perform serial dilutions to aid in the enumeration of the microbial flora, and cleaning solution(s) for rinsing and washing the digits of the individual(s). Another embodiment of a kit can include separately packaged growth medium and a selectable agent (e.g., an antibiotic) to be combined into the growth medium.

The devices, methods and kits of the present invention can be used whenever it is desired to know if a microorganism or a microbial population exists on an extremity, including digits of an individual(s). Embodiments of the invention can be used to test for the presence of bacteria, viruses, fungi, protozoa, molds, yeasts, and archaebacteria.

In some embodiments, the devices, methods and kits of the invention can be used to test for the presence of a bacterial population including but not limited to Cocci (round-shaped) bacteria, Bacilli (rod-shaped) bacteria, Spirilli (corkscrew-shaped) bacteria, pathogenic bacteria, non-pathogenic bacteria, normal flora bacteria, gram positive bacteria, gram-negative bacteria, drug-sensitive bacteria, drug-resistant bacteria, Acidaminococcus, Acinetobacter (e.g., Iwoffi), Aeromonas, Alcaligenes, Bacteroides, Bordetella, Branhamella, Brucella, Calymmatobacterium, Campylobacter, Cardiobacterium, Chromobacterium, Citrobacter (e.g., freundii), Coliform group, Edwardsiella, Enterobacter (e.g., sakazaki, aerogenes, cloacae, agglomerans), Enterococcus (e.g., faecalis, faecium), Escherichia (e.g., coli, coli-O157), Flavobacterium, Francisella, Fusobacterium, Haemophilus, Hafnia alvei, Klebsiella (e.g., oxytoca, pneumoniae), Legionella, Moraxella, Morganella (e.g. morganii), Neisseria, Pasturella, Plesiomonas, Proteus (e.g., mirabilis), Providencia, Pseudomonas (e.g., aeruginosa), Salmonella (e.g., typhimurium), Serratia (e.g., marcescens), Shigella (e.g. flexneri), Streptobacillus, Veillonella, Vibrio (e.g., cholera), Yersinia (e.g., entercolitica), Xanthomanas maltophiia, Staphylococcus (e.g., albus, aureus), Streptococcus (e.g., dysgalacticae), Micrococcus, Peptococcus, Peptostreptococcus, Bacillus (e.g., cereus), Clostridium, Lactobacillus, Listeria (e.g., monocytogenes), Erysipelothrix, Propionibacterium, Eubacterium, and Corynebacterium species, among others.

In another embodiment, the devices, methods and kits of the invention can be used to test for the presence of a fungal population including but not limited to drug-sensitive fungus, drug-resistant fungus, Candida (including C. albicans, C. tropicalis, C. parapsilosis, C. stellatoidea, C. krusei, C. parakrusei, C. lusitanae, C. pseudotropicalis, C. guilliermondi and C. glabrata), Aspergillus (including A. fumigatus, A. flavus, A. niger, A. nidulans, A. terreus, A. sydowi, A. fiavatus, and A. glaucus), Cryptococcus, Histoplasma, Coccidioides, Paracoccidioides, Blastomyces, Basidiobolus, Conidiobolus, Rhizopus, Rhizomucor, Mucor, Absidia, Mortierella, CunninghamelIa, Saksenaea, Pseudallescheria, Sporotrichosis, Fusarium, Trichophyton, Trichosporon, Microsporum, Epidermophyton, Scytalidium, Malassezia, Actinomycetes, Sporothrix, Penicillium, Saccharomyces and Pneumocystis, among others.

In yet another embodiment, the devices, methods and kits of the invention can be used to test for the presence of any viral population including but not limited to DNA virus, RNA virus, retrovirus, pathogenic virus, non-pathogenic virus, drug-resistant virus, drug-sensitive virus, adeno-associated virus, bird flu virus, cauliflower mosaic virus, Chlamydia caused by viral infection, cytomegalovirus (CMV), dengue virus, Epstein-Barr virus, feline leukemia virus, flavivirus, haemophilus influenza, hemorrhagic fever viruses, hepatitis virus (including hepatitis A, B, C, and E, viruses), herpes simplex virus, human herpesvirus type A and B, human immunodeficiency virus (HIV), human papilloma virus, human T-cell lymphotrophic virus (e.g., HTLV Type I, HTLV Type II), influenza virus, Japanese encephalitis virus, moraxella catarrhalis, non-typeable haemophilus, reovirus, parainfluenza, parvovirus, papova virus, Respiratory syncytial virus, Rubella virus, rotavirus, SARS, tomato bushy stunt virus, varicella-zoster virus, and vaccinia virus, among others.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference herein.

What is claimed is:

1. A device for sampling for the presence of a microorganism, comprising:
a container with a growth medium, wherein the growth medium is delineated into quadrants and the quadrants can be observed through the growth medium.

2. The device of claim 1, wherein the container comprises a cell culture plate or tray.

3. The device of claim 1, wherein the growth medium is selective for a microorganism selected from the group consisting of bacteria, fingi, yeast, virus, phage and mycoplasma.

4. The device of claim 3, wherein the bacteria are aerobic, anaerobic or microaerophilic.

5. The device of claim 3, wherein the bacteria are selected from the group consisting of Escherichia coli, Bacillus subtilis, Staphylococcus aureus, and Pseudomonas aeruginosa.

6. The device of claim 1, wherein the growth medium comprises a selectable agent.

7. The device of claim 6, wherein the selectable agent comprises an antibiotic or an agent having an amino acid deficiency.

8. The device of claim 1, further comprising a sheet attached to a bottom surface of the device, said sheet comprising a layout or grid to delineate said quadrants.

9. The device of claim 8, wherein the sheet delineates the medium into ten quadrants.

10. The device of claim 9, wherein the sheet comprises multiple depictions of a right hand and multiple depictions of a left hand.

11. A method for sampling for the presence of a microorganism, comprising:
placing a digit of a hand of an individual on a surface of a growth medium in a quadrant of a container, wherein the bottom surface of the container comprises a sheet with a grid or layout that delineates the growth medium into said quadrants.

12. The method of claim 11, further comprising rolling the digit from left to right.

13. The method of claim 11, wherein the sheet further comprises a depiction of a hand to indicate the placement of a digit of said hand.

14. The method of claim 11, wherein the device comprises a cell culture plate or tray.

15. The method of claim 11, wherein the growth medium is selective for a microorganism selected from the group consisting of bacteria, fungi, yeast, virus, phage and mycoplasma.

16. A kit comprising in association:
a device with growth medium;
a sheet that can be coupled to the bottom surface of the device comprising a visible layout or grid that delineates the growth medium into quadrants; and
instructions for use of the device.

17. The kit of claim 16, wherein the sheet further comprises a depiction of a hand to indicate the placement of each digit of each hand.

18. The kit of claim 16, wherein the kit further comprises a selectable agent.

19. The kit of claim 16, wherein the device comprises a cell culture plate or tray.

20. The kit of claim aim 16, wherein the growth medium is selective for a microorganism selected from the group consisting of bacteria, fungi, yeast, virus, phage and mycoplasma.
